(19) United States
(12) Patent Application Publication
(10) Pub. No.: US 2014/0328777 A1
(43) Pub. Date: Nov. 6, 2014

(54) SUNSCREENS

(71) Applicant: Christine Mendrok-Edinger, Basel (CH)

(72) Inventor: Christine Mendrok-Edinger, Basel (CH)

(73) Assignee: DSM IP ASSETS B.V., Heerlen (NL)

(21) Appl. No.: 14/124,749

(22) PCT Filed: Jun. 6, 2012
(86) PCT No.: PCT/EP2012/060671

§ 371 Date: Mar. 14, 2014

(30) Foreign Application Priority Data
Jun. 8, 2011 (EP) 11169202.6
Jun. 15, 2011 (EP) 11169926.0
Jul. 12, 2011 (EP) 11173614.6

(51) Int. Cl. C07D 249/20; A61K 31/4192; A61Q 17/04

(52) U.S. Cl. CPC C07D 249/20; A61Q 17/04; A61K 31/4192
USPC 424/60; 424/59; 548/260

(57) ABSTRACT

The present invention relates to a composition for topical application comprising at least a benzotriazol derivative, at least one organic UV-filter substance and a cosmetic solvent selected from the group of benzoate solvents such as C₁₂₋₁₅ alkyl benzoate. Furthermore, the invention relates to the use of at least one specific benzotriazol derivative for increasing the washability of UV-filter substances in topical compositions comprising a cosmetic solvent selected from the group of benzoate solvents such as C₁₂₋₁₅ alkyl benzoate.

The present invention relates to a composition for topical application comprising at least a benzotriazol derivative, at least one organic UV-filter substance and a cosmetic solvent selected from the group of benzoate solvents such as C₁₂₋₁₅ alkyl benzoate. Furthermore, the invention relates to the use of at least one specific benzotriazol derivative for increasing the washability of UV-filter substances in topical compositions comprising a cosmetic solvent selected from the group of benzoate solvents such as C₁₂₋₁₅ alkyl benzoate.

Sun care products have evolved considerably over the years. Earlier formulations were intended to protect the user from UV-B radiation as was once thought that UV-B rays were the most important contributors to wrinkling, skin disease, and skin cancer. However, more recent studies have shown that UV-A radiation is equally or even more important in the development of solar damage and skin diseases, such as lupus erythematosus and melanoma and non-melanoma skin cancer. Thus, today's focus is towards eliminating as much of UVA (320-400 nm) and/or UVB (280-320 nm) light as possible. Consequently, there's a constantly increasing need for sun care products exhibiting high SPF's (Sun Protection Factor) and high UVA protection while being photostable.

Organic UV-filter substances have excellent UV-light absorbing properties and thus contribute significantly to the SPF of a sun care product. However, they have the problem that incorporated into sun care products they tend to stain clothes which is highly undesirable. In addition, the yellow to brownish stains are not readily removable via conventional laundry.

It was therefore the object of the present invention to remedy the disadvantages of the prior art and to develop sun care products which exhibit an increased washability in order to facilitate the removal of the stains from the clothes.

Surprisingly it has been found that washability of organic UV-filter substances can be significantly improved by the addition of a cosmetic solvent selected from the group of benzoate solvents such as C₁₂₋₁₅ alkyl benzoate and specific benzotriazol derivatives.

Thus, the invention relates in one aspect to the use of at least one benzotriazol derivative of formula (I)

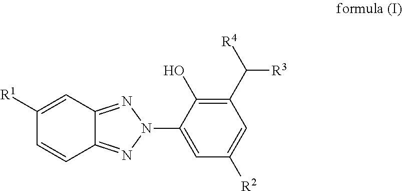

wherein

R¹ is hydrogen; C₁₋₅alkyl; C₁₋₅alkoxy or halogen; preferably hydrogen or chloride; most preferably hydrogen;

R² is hydrogen; C₁₋₂₀alkyl; C₁₋₅alkoxy; C₁₋₅alkoxycarbonyl; C₅₋₁₀cycloalkyl; C₆₋₁ₒaryl or aralkyl; preferably hydrogen or C₁₋₅alkyl; most preferably methyl;

R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and

R⁴ is hydrogen or C₁₋₅alkyl; preferably hydrogen.

for increasing the washability of organic UV-filter substances in the presence of a cosmetic solvent selected from the group of benzoate solvents such as particularly C₁₂₋₁₅ alkyl benzoate. In particular the organic UV-filter substances are incorporated into a topical composition. Such compositions are novel.

Thus, the invention also relates to a topical composition comprising a cosmetic solvent selected from the group of benzoate solvents such as particularly C₁₂₋₁₅ alkyl benzoate, at least one organic UV-filter substance and at least one benzotriazol derivative of formula (I)

In a further aspect the invention relates to a method for increasing the washability of at least one organic UV-filter substance in a topical composition comprising a cosmetic solvent selected from the group of benzoate solvents such as particularly C₁₂₋₁₅ alkyl benzoate, said method comprising the addition of at least one benzotriazol derivative of formula (I) into said topical composition and observing or appreciating the result.

The term “C_{x-y}alkyl” as used herein refers to straight-chain or branched alkyl radicals having x to y carbon atoms such as e.g. methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethyl propyl, 1-methylpentyl, 2-methyl pentyl, 3-methylpentyl, 4-methylpentyl, 1,1-dimethyl propyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,2,2-trimethylpropyl, 1-ethyl-1-methylpropyl, 1-ethyl-2-methylpropyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl without being limited thereto.

The term C₅₋₁₀cycloalkyl denotes to unsubstituted or C₁₋₁₀alkyl (mono- or poly-)substituted, such as in particular C₁₋₅alkyl (mono- or poly-)substituted cyclic, bicyclic or tricyclic hydrocarbon residues (e.g. unsubstituted or C₁₋₁₀alkyl (mono- or poly-)substituted cyclopentyl, cyclohexyl, cycoheptyl or decahydronaphtyl residues). Preferably, C₅₋₁₀cycloalkyl denotes to unsubstituted or C₁₋₂alkyl (mono- or poly-)substituted cyclopentyl, cyclohexyl or cycoheptyl residues such as in particular to unsubstituted or (mono- or poly-) methyl substituted cyclohexyl such as most in particular to cyclohexyl or 3,3,5-trimethyl-cyclohexyl.

The term “cosmetic solvent” as used herein relates to any solvent conventionally used in topical, in particular topical cosmetic compositions. The cosmetic solvents have the functional use to dissolve other ingredients such as in particular solid ingredients, e.g. solid UV-filter substances such as e.g. butyl methoxy dibenzoylmethane.

The term “benzoate solvent” refers in particular to Behenyl Benzoate (Finsolv 137), Benzyl Benzoate (Morflex Benzyl Benzoate), Butyloctyl Benzoate, C12-15 Alkyl Benzoate (Finsolv TN), C16-17 Alkyl Benzoate (Finsolv G2), Castor Oil Benzoate (Finsolv BCO-115), Cetyl Ricinoleate Benzoate (Finsolv BCR-111), C12-15 Pareth-3 Benzoate (Dermol Z5-3B), Dipentaerythrityl Hexabehenate/Benzoate/Ethylhexanoate (Nomcort H R-822), Dipropylene Glycol Dibenzoate (Finsolv PG-22, Uniflex 9-88), Ethylhexyl Benzoate (Finsolv EB), Ethylhexyl Hydroxystearate Benzoate (Finsolv BOHS-111), Hexyldecyl benzoate, Isobutyl Benzoate, Isostearyl Benzoate (Finsolv SB), Laureth-2 Benzoate (Berne) Ester 126), Lauryl/Myristyl Benzoate (Corum 5014), Linalyl Benzoate (Linalyl Benzoate), Octyldodecyl Benzoate (Finsolv BOD), Oleth-2 Benzoate, Panthenyl Ethyl Ether Benzoate, Pentaerythrityl Tetrabehenate/Benzoate/Ethyl hexanoate (Salacos P-B822), Pentaerythrityl Tetrabenzoate (Uniplex 552), Pentaerythrityl Tetraethylhexanoate/Benzoate (Salacos BO-63, Salacos P-B8(75)), Phenethyl Benzoate(X-Tend 226, Finsolv Sun), Phenyl Benzoate, PPG-15 Stearyl Ether Benzoate (Finsolv P), Propyl Benzoate, Propylene Glycol Dibenzoate (LexFeel Shine), Stearyl Benzoate (Finsolv 116). Particularly preferred in all embodiments of the invention is C₁₂₋₁₅ alkyl benzoate.

C₁₂₋₁₅ alkyl benzoate [CAS No. 68411-27-8] is e.g. commercially available as Finsolv® TN at Fintex or Innospec Active Chemicals, LLC.

The term “C_{x-y}alkoxy” as used herein denotes to linear or branched alkoxy-, respectively unsubstituted or (mono- or poly-)substituted cycloalkoxy radicals having from x to y carbon atoms such as e.g. methoxy, ethoxy, propoxy, butyloxy or pentyloxy, 2,5,5-trimethylhexyloxy, 3,5,5-trimethylhexyloxy, isoamyloxy, 2-ethylhexyloxy or 3,3,5-trimethyl-cyclohexyloxy.

The term C₆₋₁₀aryl refers e.g. to naphthyl or phenyl radicals, preferably phenyl.

The term “cloth” as used herein refers to any textile product made of materials such as cotton, wool, silk, linen, nylon, elastan, satin, polyacryl, fleece, hanf, polyester, viscose, leather as well as mixtures thereof. Particularly affected are white or lightly coloured textiles such as white T-shirts, blouses, skirts, pants, bathing textiles etc.

The term “organic UV-filter substances” refers to chemical UV-filter substances (in contrast to physical UV-filter substances such as e.g. based on metal oxides). Suitable organic UV-filter substances encompass acrylates such as e.g. 2-ethylhexyl 2-cyano-3,3-diphenylacrylate (octocrylene, PARSOL® 340), ethyl 2-cyano-3,3-diphenylacrylate; camphor derivatives such as e.g. 4-methyl benzylidene camphor (PARSOL® 5000), 3-benzylidene camphor, camphor benzalkonium methosulfate, polyacrylamidomethyl benzylidene camphor, sulfo benzylidene camphor, sulphomethyl benzylidene camphor, terephthalylidene dicamphor sulfonic acid (Mexoryl® SX); cinnamate derivatives such as e.g. ethylhexyl methoxycinnamate (PARSOL® MCX), ethoxyethyl methoxycinnamate, isoamyl methoxycinnamate as well as cinnamic acid derivatives bond to siloxanes; p-aminobenzoic acid derivatives such as e.g. p-aminobenzoic acid, 2-ethylhexyl p-dimethylaminobenzoate, N-oxypropylenated ethyl p-aminobenzoate, glyceryl p-aminobenzoate; benzophenones such as e.g. benzophenone-3, benzophenone-4,2,2′,4,4′-tetrahydroxy-benzophenone, 2,2′-dihydroxy-4,4′-dimethoxybenzophenone; esters of benzalmalonic acid such as e.g. di-(2-ethylhexyl) 4-methoxybenzalmalonate; organosiloxane compounds carrying chromophore groups such as e.g. polysilicones-15 (PARSOL® SLX), drometrizole trisiloxane (Mexoryl® XL); imidazole derivatives such as e.g. 2-phenyl benzimidazole sulfonic acid (PARSOL®HS) and salts thereof such as e.g. sodium- or potassium salts, ammonium salts, morpholine salts, salts of primary, sec. and tert. amines like monoethanolamine salts, diethanolamine salts; salicylate derivatives such as e.g. isopropylbenzyl salicylate, benzyl salicylate, butyl salicylate, ethylhexyl salicylate (PARSOL® EHS, Neo Heliopan® OS), isooctyl salicylate or homomenthyl salicylate (homosalate, PARSOL® HMS, Neo Heliopan® HMS); Triazine derivatives such as e.g. ethylhexyl triazone (Uvinul® T-150), diethylhexyl butamido triazone (Uvasorb® HEB), bis-ethylhexyloxyphenol methoxyphenyl triazine (Tinosorb® 5); Benzotriazole derivatives such as e.g. 2,2′-methylene-bis-(6-(2H-benzotriazole-2-yl)-4-(1,1,3,3,-tetramethylbutyl)-phenol (Tinosorb® M); phenylene-1,4-bis-benzimidazolsulfonic acids or salts such as e.g. 2,2-(1,4-phenylene)bis-(1H-benzimidazol-4,6-disulfonic acid) (Neoheliopan AP); benzoxazol-derivatives such as e.g. 2,4-bis-[5-1(dimethylpropyl)benzoxazol-2-yl-(4-phenyl)-imino]-6-(2-ethylhexyl)-imino-1,3,5-triazin [Uvasorb® K2A); dibenzoylmethane derivatives such as e.g. Butyl Methoxy Dibenzoylmethane; IUPAC Name: 1-(4-Methoxyphenyl)-3-(4-tert-butylphenyl)propane-1,3-dione) which is e.g. commercially available as PARSOL® 1789 at DSM Nutritional Products Ltd and amino substituted benzophenones such as Diethylamino hydroxybenzoyl hexyl benzoate [IUPAC Name: Hexyl 2-[4-(diethylamino)-2-hydroxybenzoyl]benzoate] sold under the tradename Uvinul® A plus by BASF.

Generally, the amount of each organic UV-filter substance in the compositions according to the invention is selected in the range of 0.1 to 10 wt.-%, preferably in the range of 0.2 to 7 wt.-%, most preferably in the range of 0.5 to 5 wt.-% with respect to the total weigh of the topical composition.

The total amount of organic UV-filter substances in the compositions according to the invention is preferably in the range of 1 to 40 wt.-%, preferably in the range of 5 to 30 wt.-%, in particular in the range of 10 to 30 wt.-% with respect to the total weight of the topical composition.

Preferred organic UV-filter substances encompass polysilicones-15, phenylbenzimidazol sulfonic acid, octocrylene, ethylhexyl methoxycinnamate, ethylhexyl triazone, ethyl hexylsalicylate, 4-methyl benzylidene camphor, benzophenones-3, homosalate, 2,4-Bis-{[4-(2-ethyl-hexyloxy)-2-hydroxy]-phenyl}-6-(4-methoxyphenyl)-1,3,5-triazin, 2-Hydroxy-4-methoxy-benzophenon, 2,2′-Methylene-bis-(6-(2H-benzotriazol-2-yl)-4-(1,1,3,3-tetramethyl-butyl)-phenol, butyl methoxy dibenzoylmethane and Diethylamino hydroxybenzoyl hexyl benzoate as well as mixtures thereof.

In one particular advantageous embodiment the organic UV-filter substances are oil soluble such as preferably UV-filter substances selected from the group consisting of ethylhexyl methoxycinnamate, ethylhexyl triazone, ethyl hexylsalicylate, butyl methoxy dibenzoylmethane and diethylamino hydroxybenzoyl hexyl benzoate and or homosalate.

In another particular advantageous embodiment the organic UV-filter substances are water soluble such as preferably phenylbenzimidazol sulfonic acid. Phenylbenzimidazolsulfonic acid is preferably used in an amount ranging from 1 to 5 wt.-%, preferably in the range of 2 to 4 wt.-%, based on the total weight of the composition.

The amount of the at least one benzotriazol derivative of formula (I) in the compositions according to the invention is at least 0.01 wt.-%. In particular, the amount of the at least one benzotriazol derivative of formula (I) in the compositions according to the invention is selected in the range of 1 to 20 wt.-%, such as in the range of 2 to 20 wt.-%, and preferably in the range of 2 to 15 wt.-%, more preferably in the range of 4 to 12 wt.-%, and most preferably in the range of 6 to 10 wt.-%, based on the total weight of the composition.

In a particular embodiment of the present invention the benzotriazol derivative is selected from compounds of formula (I) wherein R¹ and R⁴ are hydrogen, R² is methyl and R³ is C₆₋₁₀alkoxy such as preferably C₆₋₁₀alkoxy, or C₆cycloalkoxy such as in particular 2,5,5-trimethylhexyloxy, 3,5,5-trimethylhexyloxy, isoamyloxy, 2-ethylhexyloxy or 3,3,5-trimethyl-cyclohexyloxy. Such compounds and their preparation are e.g. disclosed in EP Application No.: 10150832.3 (PCT publication: WO2011086124). Particularly preferred according to the present invention is a compound of formula (I), wherein R¹ and R⁴ are hydrogen, R² is methyl and R³ is 2-ethylhexyloxy (i.e. 2-(2H-Benzotriazol-2-yl)-6-(2-ethylhexyloxymethyl)-4-methyl-phenol).

In another particular embodiment of the present invention the compound of formula (I) is a compound wherein R¹ and R⁴ are hydrogen, R² is methyl and R³ is undecyl (C₁₁H₂₃) which is commercially available as Tinogard TL [INCI Name: benzotriazolyl dodecyl p-cresol; IUPAC Name: 2-(2H-benzotriazol-2-yl)-6-dodecyl-4-methyl-phenol] at BASF SE Ludwigshafen.

The amount of the benzoate solvent such as in particular the C₁₂₋₁₅ alkyl benzoate in the compositions according to the invention is preferable selected in the range of 1 to 50 wt.-%, in particular in the range of 5 to 30 wt.-%, such as about 10 to 30 wt.-%, based on the total weight of the composition.

In a particular embodiment according to the present invention, the topical compositions according to the present invention comprise besides the C₁₂₋₁₅ alkyl benzoate no further cosmetic solvents.

In a further particular embodiment the compositions according to the present invention comprise homomenthyl salicylate (e.g. commercially available as PARSOL HMS at DSM Nutritional Products Ltd Kaiseraugst) in combination with at least one further organic UV-filter substance with the definitions and preferences as given above. The homomenthyl salicylate (INCI homosalate) is preferably used in an amount ranging from 1 to 25 wt.-% such as in an amount of 5 to 15 wt.-% such as about 10 wt.-%, based on the total weight of the composition.

In another particular embodiment the compositions according to the present invention are substantially free of a polyglycerol based UV-filter such as e.g. disclosed in [EP Application No's] EP09178503.0, EP09178501.4, EP09178502.2 EP09178495.9, EP09178506.3, EP09178505.5 or EP10150832.3 which are obtainable by a process comprising the steps of ring-opening polymerization of x mol equivalents of glycidol using 1 mol equivalent of a polyol starter unit with y mol equivalents hydroxyl-groups, followed by block copolymerization with z×(x+y) mole equivalents of propylene oxide to form a hyperbranched polyether-polyol backbone carrying (x+y) mol equivalents hydroxyl-groups followed by partial or total esterification, respectively partial or total etherification of the hydroxyl groups with a UV-light absorbing chromophore such as particularly with p-dimethylamino benzoic acid, 3-[1-(4-Hydroxymethyl-phenyl)-meth-(E)-ylidene]-1,7,7-trimethyl-bicyclo[2.2.1]heptan-2-one, 2-(4-diethylamino-2-hydroxybenzoyl)benzoic acid, p-alkoxycinnamic acid, 2-cyano-3,3-diphenylacrylic acid as well as mixtures thereof and wherein x is an integer selected in the range from 3-16, y is an integer selected in the range from 1-6, and z is an integer selected in the range from 0-10.

The term “topical” is understood here to mean external application to keratinous substances, which are in particular the skin, scalp, eyelashes, eyebrows, nails, mucous membranes and hair.

As the compositions according to the invention are intended for topical application, they comprise a physiologically acceptable medium, that is to say a medium compatible with keratinous substances, such as the skin, mucous membranes, and keratinous fibres. In particular the physiologically acceptable medium is a cosmetically acceptable carrier.

The term cosmetically acceptable carrier refers to all carriers and/or excipients and or diluents conventionally used in cosmetic compositions.

Preferred topical compositions according to the invention are skin care preparations, decorative preparations, and functional preparations.

Examples of skin care preparations are, in particular, light protective preparations, anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples of decorative preparations are, in particular, lipsticks, eye shadows, mascaras, dry and moist make-up formulations, rouges and/or powders.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment the topical compositions according to the invention are light-protective preparations (sun care products), such as sun protection milks, sun protection lotions, sun protection creams, sun protection oils, sun protection sprays, sun blocks or tropical's or day care creams with a SPF (sun protection factor). Of particular interest are sun protection creams, sun protection lotions, sun protection sprays, sun protection milks and sun protection preparations.

The topical compositions according to the present invention may be in the form of a suspension or dispersion in solvents or fatty substances, or alternatively in the form of an emulsion or micro emulsion (in particular of oil-in-water (O/W-) or water-in-oil (W/O-)type, silicone-in-water (Si/W-) or water-in-silicone (W/Si-)type, PIT-emulsion, multiple emulsion (e.g. oil-in-water-in oil (O/W/O-) or water-in-oil-in-water (W/O/W-)type), pickering emulsion, hydrogel, alcoholic gel, lipogel, one- or multiphase solution or vesicular dispersion or other usual forms, which can also be applied by pens, as masks or as sprays (e.g. as “emulsion sprays and clear sprays, both available as pump sprays and continuous sprays (aerosols)”).

The topical compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art and illustrated in the examples.

If the topical composition according to the invention is an O/W emulsion, then it contains advantageously at least one O/W- or Si/W-emulsifier selected from the list of PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10Polyoxyl 10 Oleyl Ether, Ceteth-10, Iosteareth-20, Ceteareth-20, Oleth-20, Steareth-20, Steareth-21, Ceteth-20, Isoceteth-20, Laureth-23, Steareth-100, glycerylstearatcitrate, glycerylstearate (self emulsifying), stearic acid, salts of stearic acid acid, polyglyceryl-3-methylglycosedistearate. Further suitable emulsifiers are phosphate esters and the salts thereof such as cetyl phosphate (Amphisol® A), diethanolamine cetyl phosphate (Amphisol®DEA), potassium cetyl phosphate (Amphisol® K), sodiumcetearylsulfat, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, Lauryl Glucoside, Decyl Glucoside, Sodium Stearoyl Glutamate, Sucrose Polystearate and Hydrated Polyisobuten. Furthermore, one or more synthetic polymers may be used as an emulsifier.

For example, PVP eicosene copolymer, acrylates/C₁₀₋₃₀ alkyl acrylate crosspolymer, acrylates/steareth-20 methacrylate copolymer, PEG-22dodecyl glycol copolymer, PEG-45/dodecyl glycol copolymer, and mixtures thereof.

The at least one O/W respectively Si/W emulsifier is preferably used in an amount of 0.5 to 10 wt.-% such as in particular in the range of 0.5 to 5 wt.-% such as most in particular in the range of 1 to 4 wt.-% based on the total weight of the composition.

Particular suitable O/W emulsifiers according to the present invention encompass phosphate esters emulsifier of formula (II)

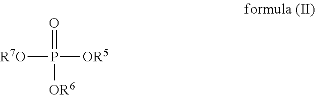

wherein R⁵, R⁶ and R⁷ may be hydrogen, an alkyl of from 1 to 22 carbons, preferably from 12 to 18 carbons; or an alkoxylated alkyl having 1 to 22 carbons, preferably from 12 to 18 carbons, and having 1 or more, preferably from 2 to 25, most preferably 2 to 12, moles ethylene oxide, with the provision that at least one of R⁵, R⁶ and R⁷ is an alkyl or alkoxylated alkyl as previously defined but having at least 6 alkyl carbons in said alkyl or alkoxylated alkyl group.

Monoesters in which R⁵ and R⁶ are hydrogen and R⁷ is selected from alkyl groups of 10 to 18 carbons and alkoxylated fatty alcohols of 10 to 18 carbons and 2 to 12 moles ethylene oxide are preferred. Among the preferred phosphate ester emulsifier are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C₁₂₋₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. A particular advantageous phosphate ester emulsifier according to the invention is potassium cetyl phosphate e.g. commercially available as Amphisol® K at DSM Nutritional Products Ltd Kaiseraugst as the overall washability is increased.

Further suitable O/W emulsifiers are polyethyleneglycol (PEG) esters or diesters such as e.g. [INCI Names], PEG-40 Stearate, PEG-40 Sorbitan Peroleate, PEG-7 Glyceryl Cocoate, PEG-20 Almond Glycerides, PEG-25 Hydrogenated Castor Oil, PEG-7 Olivate, PEG-8 Oleate, PEG-8 Laurate, PEG-8 Dilaurate, PEG-8 Stearate, PEG-60 Almond Glycerides, PEG-20 Methyl Glucose Sesquistearate, PEG-100 Stearate, PEG-80 Sorbitan Laurate. Particularly preferred according to the present invention is PEG-100 Stearate e.g. sold under the tradename Myrj S100 by Croda or in combination with Glyceryl stearate under the tradename Arlacel™ 165 (INCI Glyceryl Stearate (and) PEG-100 Stearate) by Croda.

Other suitable O/W emulsifiers are anionic polymeric 0W emulsifiers such as e.g. a block copolymer consisting of a poly acrylic acid such as e.g. hydrophobically modified polyacrylic acid (i.e. AcrylatesC10-30 Alkyl Acrylate Crosspolymer) produced by Noveon Inc and sold as Pemulen® TR-1 or TR2.

Other suitable O/W emulsifiers are polyethoxylated alcohols such as eg. Steareth-2 and Steareth-21, Steareth-10, Steareth-11, Steareth-13, Steareth-14, Steareth-15, Steareth-16, Steareth-20, Steareth-25, Steareth-27, Steareth-30, Steareth-40, Steareth-50, Steareth-80, Steareth-100, Steareth-200, Isosteareth-20. Particularly preferred according to the present invention is Steareth-2 and Steareth-21 sold under the trade names Brij 72 (INCI: Steareth-2) and Brij 721 (INCI: Steareth-21).

Other suitable O/W emulsifiers are glucosides such as Cetearyl Glucoside, C10-16 Alkyl Glucoside, C12-18 Alkyl Glucoside, C12-20 Alkyl Glucoside, C20-22 Alkyl Glucoside, Butylglucoside Caprate, Caproyl Ethyl Glucoside, CaprylylCapryl Glucoside, Caprylyl Glucoside, Coco-Glucoside, Cocoyl Ethyl Glucoside, Decyl Glucoside, Ethyl Glucoside, Lauryl Glucoside. Particularly preferred according to the present invention is Cetearyl Glucoside sold under the trade name Tego Care CG 90 (INCI: Cetearyl Glucoside).

Further suitable emulsifiers according to the present invention are W/O emulsifiers such as in particular are polyethyleneglycol (PEG) esters or diesters such as PEG-30 Dipolyhydroxystearate, PEG-2 Dilaurate, PEG-4 Dilaurate, PEG-6 Dilaurate, PEG-8 Dioleate, PEG-4 Dicocoate, PEG-4 Diisostearate. Particularly preferred according to the present invention is PEG-30 Dipolyhydroxystearate sold under the trade name Cithrol DPHS (INCI: PEG-30 Dipolyhydroxystearate).

In particular embodiment, the topical compositions according to the present invention are in the form of O/W emulsions comprising an oily phase dispersed in an aqueous phase in the presence of an O/W emulsifier, wherein the benzotriazol derivative of formula (I) is benzotriazolyl dodecyl p-cresol, the organic UV-filter substance is selected from the group consisting of ethylhexyl methoxycinnamate, ethylhexyl triazone, ethyl hexylsalicylate, butyl methoxy dibenzoylmethane, diethylamino hydroxybenzoyl hexyl benzoate, phenylbenzimidazol sulfonic acid and homosalate as well as mixtures thereof, and the O/W emulsifier is potassium cetyl phosphate.

In another particular embodiment, the topical compositions according to the present invention are in the form of O/W emulsions comprising an oily phase dispersed in an aqueous phase in the presence of an O/W emulsifier wherein the benzotriazol derivative of formula (I) is a compound of formula (I) wherein R¹ and R⁴ are hydrogen, R² is methyl and R³ is 2,5,5-trimethylhexyloxy, 3,5,5-trimethylhexyloxy, isoamyloxy, 2-ethylhexyloxy or 3,3,5-trimethyl-cyclohexyloxy such as in 2-(2H-Benzotriazol-2-yl)-6-(2-ethylhexyloxymethyl)-4-methyl-phenol, the organic UV-filter substance is selected from the group consisting of ethylhexyl methoxycinnamate, ethylhexyl triazone, ethyl hexylsalicylate, butyl methoxy dibenzoylmethane, diethylamino hydroxybenzoyl hexyl benzoate, phenylbenzimidazol sulfonic acid and homosalate as well as mixtures thereof, and the O/W emulsifier is potassium cetyl phosphate.

The topical compositions according to the present invention furthermore advantageously contain at least one co-surfactant such as e.g. selected from the group of mono- and diglycerides and or fatty alcohols. The co-surfactant is generally used in an amount selected in the range of 0.1 to 10 wt.-%, such as in particular in the range of 0.5 to 5 wt.-%, such as most in particular in the range of 1 to 3 wt.-%, based on the total weight of the composition. Particular suitable co-surfactants are selected from the list of alkyl alcohols such as cetyl alcohol (Lorol C16, Lanette 16) cetearyl alcohol (Lanette 0), stearyl alcohol (Lanette 18), behenyl alcohol (Lanette 22), glyceryl stearate, glyceryl myristate (Estol 3650), hydrogenated coco-glycerides (Lipocire Na10) as well as mixtures thereof.

The compositions in form of O/W emulsions according to the invention can be provided, for example, in all the formulation forms for O/W emulsions, for example in the form of serum, milk, spray or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the compositions constitute cosmetic composition and are intended for topical application to the skin.

In a particular embodiment, the use and methods according to the present invention refer to an increased washability of the UV-filter substances with the definitions and preferences as given above from clothes and or the skin. In particular the clothes are white or light colored clothes.

Finally, a subject-matter of the invention is a method for the cosmetic treatment of keratinous substances such as in particular the skin, characterized in that a composition as defined above is applied to the said keratinous substances such as in particular to the skin. The method is in particular suitable to protect the skin against the adverse effects of UV-radiation such as in particular sun-burn and or photoageing.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person. The additional ingredients can either be added to the oily phase, the aqueous phase or separately as deemed appropriate. The mode of addition can easily be adapted by a person skilled in the art.

The cosmetically active ingredients useful herein can in some instances provide more than one benefit or operate via more than one mode of action.

The topical cosmetic compositions of the invention can also contain usual cosmetic adjuvants and additives, such as preservatives antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention are e.g. described in the CTFA Cosmetic Ingredient Handbook, Second Edition (1992), The Cosmetic, Toiletry and Fragrance Association, Inc or International Cosmetic Ingredient Dictionary and Handbook, Vol. 4, 2008 (ISBN-10: 1882621433) without being limited thereto.

In a further particular embodiment, the compositions according to the present invention furthermore comprise a high molecular weight polysaccharide such as in particular Xanthan gum (e.g. available under the tradename Keltrol CG at Rahn AG Zurich). The Xanthan gum is preferably used in the compositions according to the present invention in an amount of 0.01 to 1 wt.-% such as in particular in an amount of 0.1 to 0.5 wt.-%, based on the total weight of the composition.

It is furthermore advantageous if the compositions according to the present invention comprise a preservative. Suitable preservatives are well known to a person skilled in the art and encompass e.g. parabens (such as methylparaben, ethylparaben, butylparaben, propylparaben and/or isobutylparaben), phenoxyethanol as well as mixtures thereof (e.g. commercially available as Phenonip at Clariant). The amount of the preservative can easily be determined by a person skilled in the art and is e.g. selected in the range of 0.1 to 2 wt.-%, based on the total weight of the composition.

The necessary amounts of the cosmetic and dermatological adjuvants and additives can based on the desired product easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The topical compositions according to the invention in general have a pH in the range of 3 to 10, preferably a pH in the range of 4 to 8 and most preferably a pH in the range of 4 to 7. The pH can easily be adjusted as desired with suitable acids such as e.g. citric acid or bases such as NaOH, Triethanolamine (TEA Care), Tromethamine (Trizma Base) and Aminomethyl Propanol (AMP-Ultra PC 2000) according to standard methods in the art.

The following examples are provided to further illustrate the compositions and effects of the present invention. These examples are illustrative only and are not intended to limit the scope of the invention in any way.

### EXAMPLE

0.75 mg/cm² of the respective compositions as outlined in table l and 2 were applied to PMMA plates and the plates were dried at RT for 30 min. Afterwards the initial in vitro SPF (Sun Protection Factor) were determined with a “m.u.t HELIAS fiber optical integrator”. Then the plates were immersed in a flask filled with 2.5 l water (bi-destilled) for 20 min while the water was stirred with a magnetic stirrer at 450 1 min at RT (The plates were attached at the edge of the flask with a clothespin, such that the side covered with the composition was directed into the flask). Afterwards the plates were dried at 40° C. for 30 min. The immersion/drying procedure was repeated once. After final drying the in-vitro SPF was measured again and the water resistance was calculated as % WR =[(SPF after immersion−1) (SPF initial −1)]*100.

Each composition was tested separately on 4 plates.

The water resistance for each composition was determined as mean value from the 4 plates.

The washability increase was calculated as {[(water resistance reference)−(water resistance sample)]/water resistance reference}*100.

(Reference samples=A; Sample according to the invention=B)

TABLE 1
Oil solutions in C₁₂₋₁₅ alkyl benzoate

| | 1A | 1B | 2A | 2B | 3A | 3B | 4A | 4B |
|---|---|---|---|---|---|---|---|---|
| Tradename | Wt.-% | | Wt.-% | | Wt.-% | | Wt.-% | |
| Finsolv ® TN | 84 | 74 | 86 | 76 | 86 | 76 | 80 | 70 |
| Parsol ® HMS | 10 | 10 | 10 | 10 | 10 | 10 | 10 | 10 |
| Tinogard ® TL | | 10 | | 10 | | 10 | | 10 |
| Parsol ® EHS | 6 | 6 | | | | | | |
| Parsol ® 1789 | | | 4 | 4 | | | | |
| Uvinul ® T150 | | | | | 4 | 4 | | |
| Parsol ® MCX | | | | | | | 10 | 10 |
| in vitro SPF initial | 5.9 | 36.4 | 14.25 | 59.7 | 8.6 | 55.9 | 12.4 | 64.7 |
| in vitro SPF after immersion | 3.5 | 14.7 | 6.8 | 20.8 | 6.5 | 23.6 | 9.4 | 29.4 |
| Water Resistance | 51% | 39% | 44% | 34% | 72% | 41% | 74% | 45% |
| Washability increase | | +24% | | +23% | | +43% | | +39% |

TABLE 2
O/W formulations with Amphisol K

| | 5A | 5B | 6A | 6B | 6C | 7A | 7B | 7C | 8A | 8B |
|---|---|---|---|---|---|---|---|---|---|---|
| Tradename | Wt-% | | Wt.-% | | | Wt.-% | | | Wt.-% | |
| Finsolv ® TN | 25 | 25 | 25 | 25 | 25 | 25 | 25 | 25 | 25 | 25 |
| Parsol ® HMS | 10 | 10 | 10 | 10 | 10 | 10 | 10 | 10 | 10 | 10 |
| Tinogard ® TL | | 10 | | 10 | | | 10 | | | 10 |
| 2-(2H-Benzo- | | | | | 10 | | | 10 | | |
| triazol-2-yl)-6- | | | | | | | | | | |
| (2-ethylhexyl- | | | | | | | | | | |
| oxy-methyl)-4- | | | | | | | | | | |
| methyl-phenol* | | | | | | | | | | |
| Parsol ® EHS | 6 | 6 | | | | | | | | |
| Parsol ® 1789 | | | 4 | 4 | 4 | | | | | |
| Uvinul ® T150 | | | | | | 4 | 4 | 4 | | |
| Parsol ® MCX | | | | | | | | | 10 | 10 |
| Amphisol ® K | 2.5 | 2.5 | 2.5 | 2.5 | 2.5 | 2.5 | 2.5 | 2.5 | 2.5 | 2.5 |
| Lanette ® O | 1.5 | 1.5 | 1.5 | 1.5 | 1.5 | 1.5 | 1.5 | 1.5 | 1.5 | 1.5 |
| Phenonip ® | 0.8 | 0.8 | 0.8 | 0.8 | 0.8 | 0.8 | 0.8 | 0.8 | 0.8 | 0.8 |
| Keltrol ® CG-T | 0.3 | 0.3 | 0.3 | 0.3 | 0.3 | 0.3 | 0.3 | 0.3 | 0.3 | 0.3 |
| Water | 53.9 | 43.9 | 55.9 | 45.9 | 45.9 | 55.9 | 45.9 | 45.9 | 49.9 | 39.9 |
| in vitro SPF | 4.7 | 23.6 | 7.7 | 25 | 15.8 | 6.9 | 29.6 | 19.1 | 10.4 | 48.8 |
| initial in | 3.2 | 12.8 | 5.7 | 12.2 | 8.8 | 5 | 14.5 | 11 | 8.2 | 28.9 |
| vitro SPF after | | | | | | | | | | |
| immersion | | | | | | | | | | |
| Water Resistance | 59% | 52% | 70% | 47% | 53% | 68% | 47% | 55% | 77% | 58% |
| Washability | | +12% | | +32% | +24% | | +31% | +19% | | +25% |
| increase | | | | | | | | | | |

| | |
|---|---|
| 9A | 9B |

| | |
|---|---|
| Tradename | Wt.-% |

| | | |
|---|---|---|
| Parsol ® HMS | 10 | 10 |
| Finsolv ® TN | 10 | 10 |
| Tinogard ® TL | | 10 |
| Amphisol ® K | 2.5 | 2.5 |
| Lanette ® O | 1.5 | 1.5 |
| Phenonip ® | 0.8 | 0.8 |
| Keltrol ® CG-T | 0.3 | 0.3 |
| Water | 68.5 | 58.5 |
| Parsol ® HS | 4 | 4 |
| Triethanolamine Care | 2.4 | 2.4 |
| in vitro SPF initial | 5.3 | 17.7 |
| in vitro SPF after immersion | 1.9 | 3.8 |
| Water Resistance | 21% | 17% |
| Washability increase | | +19% |

*benzotriazol derivative of formula (I): 2-(2H-Benzotriazol-2-yl)-6-(2-ethylhexyloxymethyl)-4-methyl-phenol (i.e. a compound of formula (I), wherein R¹ and R⁴ are hydrogen, R² is methyl and R³ is 2-ethylhexyloxy)

TABLE 3
Pemulen gel

| | 10A | 10B | 11A | 11B | 12A | 12B | 13A | 13B |
|---|---|---|---|---|---|---|---|---|
| Tradename | Wt.-% | | Wt.-% | | Wt.-% | | Wt.-% | |
| Finsolv ® TN | 25 | 25 | 25 | 25 | 25 | 25 | 25 | 25 |
| Parsol ® HMS | 10 | 10 | 10 | 10 | 10 | 10 | 10 | 10 |
| Tinogard ® TL | | 10 | | 10 | | 10 | | 10 |
| Parsol ® EHS | 6 | 6 | | | | | | |
| Parsol ® 1789 | | | 4 | 4 | | | | |
| Uvinul ® T150 | | | | | 4 | 4 | | |
| Parsol ® MCX | | | | | | | 10 | 10 |
| Pemulen ™ TR2 | 0.4 | 0.4 | 0.4 | 0.4 | 0.4 | 0.4 | 0.4 | 0.4 |
| Phenonip ® | 0.8 | 0.8 | 0.8 | 0.8 | 0.8 | 0.8 | 0.8 | 0.8 |
| Keltrol ® CG-T | 0.3 | 0.3 | 0.3 | 0.3 | 0.3 | 0.3 | 0.3 | 0.3 |
| Triethanolamine Care | 0.4 | 0.4 | 0.4 | 0.4 | 0.4 | 0.4 | 0.4 | 0.4 |
| Water | 57.1 | 47.1 | 59.1 | 49.1 | 59.1 | 49.1 | 53.1 | 43.1 |
| in vitro SPF initial | 4.8 | 22.1 | 7.5 | 27.8 | 6.3 | 20.5 | 9.4 | 38.7 |
| in vitro SPF after immersion | 3.5 | 12.6 | 5.5 | 15.8 | 4.8 | 12.7 | 7.4 | 22.8 |
| Water Resistance | 66% | 55% | 69% | 55% | 72% | 60% | 76% | 58% |
| Washability increase | | +11 | | +14 | | +8 | | +18 |

O/W formulation with Arlacel 165

| | | | |
|---|---|---|---|
| 14A | 14B | 15A | 15B |

| | | |
|---|---|---|
| Tradename | Wt.-% | Wt.-% |

| | | | | |
|---|---|---|---|---|
| Finsolv ® TN | 25 | 25 | 25 | 25 |
| Parsol ® HMS | 10 | 10 | 10 | 10 |
| Tinogard ® TL | | 10 | | 10 |
| Parsol ® 1789 | 4 | 4 | | |
| Uvinul ® T150 | | | 4 | 4 |
| Arlacel ® 165 | 5 | 5 | 5 | 5 |
| Lanette ® O | 1.5 | 1.5 | 1.5 | 1.5 |
| Phenonip ® | 0.8 | 0.8 | 0.8 | 0.8 |
| Keltrol ® CG-T | 0.3 | 0.3 | 0.3 | 0.3 |
| Water | 53.4 | 43.4 | 53.4 | 43.4 |
| in vitro SPF initial | 9.4 | 31.5 | 7.6 | 17.6 |
| in vitro SPF after immersion | 6.2 | 18.6 | 6.8 | 13.2 |
| Water Resistance | 62% | 58% | 88% | 73% |
| Washability increase* | | +4% | | +15% |

TABLE 4
O/W formulation with Brij and C₁₂₋₁₅ alkyl benzoate

| | | | |
|---|---|---|---|
| 16A | 16B | 17A | 17B |

| | | |
|---|---|---|
| Tradename | Wt.-% | Wt.-% |

| | | | | |
|---|---|---|---|---|
| Finsolv ® TN | 25 | 25 | 25 | 25 |
| Parsol ® HMS | 10 | 10 | 10 | 10 |
| Tinogard ® TL | | 10 | | 10 |
| Parsol ® EHS | 6 | 6 | | |
| Parsol ® MCX | | | 10 | 10 |
| Brij ® 72 | 2.5 | 2.5 | 2.5 | 2.5 |
| Brij ® 721 | 2.5 | 2.5 | 2.5 | 2.5 |
| Lanette ® O | 1 | 1 | 1 | 1 |
| Phenonip ® | 0.8 | 0.8 | 0.8 | 0.8 |
| Keltrol ® CG-T | 0.3 | 0.3 | 0.3 | 0.3 |
| Water | 51.9 | 41.9 | 47.9 | 37.9 |
| in vitro SPF initial | 4.5 | 22.9 | 11.6 | 38.6 |
| in vitro SPF after immersion | 3.2 | 14.4 | 9.1 | 28 |
| Water Resistance | 63% | 61% | 76% | 72% |
| Washability increase | | +2% | | +4% |

TABLE 5
W/O formulation with Cithrol DPHS (PEG-30 Dipolyhydroxystearate)

| | 18A | 18B | 19A | 19B | 20A | 20B | 21A | 21B |
|---|---|---|---|---|---|---|---|---|
| Tradename | Wt.-% | | Wt.-% | | Wt.-% | | Wt.-% | |
| Finsolv ® TN | 25 | 25 | 25 | 25 | 25 | 25 | 25 | 25 |
| Parsol ® HMS | 10 | 10 | 10 | 10 | 10 | 10 | 10 | 10 |
| Tinogard TL | | 10 | | 10 | | 10 | | 10 |
| Parsol ® EHS | 6 | 6 | | | | | | |
| Uvinul ® T150 | | | 4 | 4 | | | | |
| Parsol ® MCX | | | | | 10 | 10 | | |
| Parsol ® HS | | | | | | | 4 | 4 |
| Cithrol ™ DPHS | 4 | 4 | 4 | 4 | 4 | 4 | 4 | 4 |
| Monomuls ® 90-O 18 | 1.5 | 1.5 | 1.5 | 1.5 | 1.5 | 1.5 | 1.5 | 1.5 |
| Triethanolamin Care | | | | | | | 2.4 | 2.4 |
| Sodiumchloride | 1 | 1 | 1 | 1 | 1 | 1 | 1 | 1 |
| Water | 52.5 | 42.5 | 54.5 | 44.5 | 48.5 | 38.5 | 52.1 | 42.1 |
| in vitro SPF initial | 4.5 | 17.8 | 6.8 | 41.2 | 9.8 | 53.8 | 7.7 | 38.2 |
| in vitro SPF after immersion | 3.8 | 14 | 5.7 | 29.5 | 8.7 | 38.8 | 7.1 | 28.1 |
| Water Resistance | 80% | 77% | 81% | 71% | 88% | 72% | 91% | 73% |
| Washability increase | | +13% | | +10% | | +16% | | +18% |

TABLE 6
Tego ® Care CG90 (Cetearyl Glucoside)

| | | | |
|---|---|---|---|
| 22A | 22B | 23A | 23B |

| | | |
|---|---|---|
| Tradename | Wt.-% | Wt.-% |

| | | | | |
|---|---|---|---|---|
| Finsolv ® TN | 25 | 25 | 25 | 25 |
| Parsol ® HMS | 10 | 10 | 10 | 10 |
| Tinogard ® TL | | 10 | | 10 |
| Parsol ® EHS | 6 | 6 | | |
| Uvinul ® T150 | | | 4 | 4 |
| Tego ® Care CG90 | 1.5 | 1.5 | 1.5 | 1.5 |
| Lanette ® O | 1 | 1 | 1 | 1 |
| Phenonip ® | 0.8 | 0.8 | 0.8 | 0.8 |
| Keltrol ® CG-T | 0.3 | 0.3 | 0.3 | 0.3 |
| Water | 55.4 | 45.4 | 57.4 | 47.4 |
| in vitro SPF initial | 4.8 | 24 | 6.4 | 32.5 |
| in vitro SPF after | 3.4 | 13.7 | 5.3 | 24.4 |
| immersion | | | | |
| Water Resistance | 63% | 55% | 80% | 74% |
| Washability increase | | +8% | | +7% |

TABLE 7
Reference Formulations caprylic/capric triglyceride (Myritol ® 318)
respectively diisopryl sebacate (DUB DIS)

| | Ref1A | Ref1B(1) | Ref1B(2) | Ref2A | Ref2B(1) |
|---|---|---|---|---|---|
| Tradename | wt.-% | | | wt.-% | |
| Myritol ® 318 | 25 | 25 | 25 | | |
| DUB DIS | | | | 25 | 25 |
| Parsol ® HMS | 10 | 10 | 10 | 10 | 10 |
| Tinogard ® TL | | 10 | | | 10 |
| 2-(2H-Benzo-triazol-2-yl)-6- | | | 10 | | |
| (2-ethylhexyl-oxy-methyl)- | | | | | |
| 4-methyl-phenol* | | | | | |
| Parsol ® 1789 | 4 | 4 | 4 | 4 | 4 |
| Amphisol ® K | 2.5 | 2.5 | 2.5 | 2.5 | 2.5 |
| Lanette ® O | 1.5 | 1.5 | 1.5 | 1.5 | 1.5 |
| Phenonip ® | 0.8 | 0.8 | 0.8 | 0.8 | 0.8 |
| Keltrol ® CG-T | 0.3 | 0.3 | 0.3 | 0.3 | 0.3 |
| Water | Ad 100 | | | Ad 100 | |
| in vitro SPF | 7.4 | 29.6 | 19.1 | 7.4 | 29.6 |
| initial | | | | | |
| in vitro SPF | 3.6 | 17.7 | 9.4 | 3.6 | 17.7 |
| after immersion | | | | | |
| Water Resistance | 41% | 58% | 46% | 41% | 58% |
| Washability | | −17% | −5% | | −17% |
| increase | | | | | |

*benzotriazol derivative of formula (I): 2-(2H-Benzotriazol-2-yl)-6-(2-ethylhexyloxymethyl)-4-methyl-phenol (i.e. a compound of formula (I), wherein R¹ and R⁴ are hydrogen, R² is methyl and R³ is 2-ethylhexyloxy)

The results shown in table 1 to 6 illustrate that the addition of a benzotriazol derivative according to the present invention significantly increases the washability of organic UV-filter substances in formulations comprising a benzoate solvent such as Finsolv TN, whereas the use of another solvent such as diisopryl sebacate (DUB DIS) and caprylic/capric triglyceride (Myritol® 318) leads to an decreased washability (table 7).

1. A topical composition comprising a cosmetic solvent selected from the group of benzoate solvents, at least one organic UV-filter substance and at least one benzotriazol derivative of formula (I) 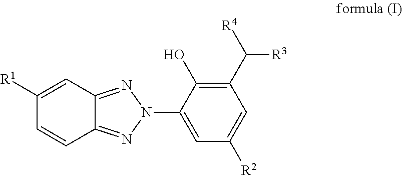
wherein
R¹ is hydrogen; C₁₋₅alkyl; C₁₋₅alkoxy or halogen; preferably hydrogen or chloride, most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl, preferably hydrogen.

2. The topical composition according to claim 1, characterized in that the benzoate solvent is C₁₂₋₁₅ alkyl benzoate.

3. The topical composition according to claim 1, characterized in that the benzotriazol derivative of formula (I) is used in an amount selected in the range of 1 to 20 wt.-% based on the total weight of the composition.

4. The topical composition according to claim 1, characterized in that the benzotriazol derivative is used in an amount selected in the range of 2 to 20 wt.-% based on the total weight of the composition.

5. The topical composition according to claim 1, characterized in that the benzotriazol compound of formula (I) is a compound wherein R¹ and R⁴ are hydrogen, R² is methyl and R³ is 2,5,5-trimethylhexyloxy, 3,5,5-trimethylhexyloxy, isoamyloxy, 2-ethylhexyloxy or 3,3,5-trimethyl-cyclohexylox or undecyl.

6. The topical composition according to claim 1, characterized in that the total amount of organic UV-filter substances is selected in the range of 1 to 40 wt.-%, based on the total weight of the composition.

7. The topical composition according to claim 1, characterized in that the organic UV-filter substance is selected from the group consisting of ethylhexyl methoxycinnamate, ethylhexyl triazone, ethyl hexylsalicylate, butyl methoxy dibenzoylmethane and diethylamino hydroxybenzoyl hexyl benzoate, homosalate and phenyl benzimidazole sulfonic acid as well as mixtures thereof.

8. The topical composition according to claim 1, characterized in that the amount of the benzoate solvent, preferably C₁₂₋₁₅ alkyl benzoate, is selected in the range of 1 to 50 wt.-%, based on the total weight of the composition.

9. The topical composition according to claim 1, characterized in that the topical composition is an O/W emulsion comprising an oily phase dispersed in an aqueous phase in the presence of an O/W emulsifier.

10. The topical composition according to claim 9, characterized in that the O/W emulsifier is selected from the group consisting of phosphate esters, PEG-100 Stearate, Glyceryl Stearate (and) PEG-100 Stearate, Steareth-2, Steareth-21, anionic polymeric O/W emulsifiers as well as mixtures thereof.

11. The topical composition according to claim 9, characterized in that the O/W emulsifier is selected from the group of phosphate esters.

12. The topical composition according to claim 9, characterized in that the amount of O/W emulsifier is selected in the range of 0.5 to 10 wt.-%, based on the total weight of the composition.

13. The topical composition according to claim 1, characterized in that the composition comprises at least one co-surfactant in an amount selected in the range of 0.1 to 10 wt.-%, based on the total weight of the composition.

14. The topical composition according to claim 13, characterized in that the co-surfactant is selected from the group consisting of cetyl alcohol, cetearyl alcohol, stearyl alcohol, behenyl alcohol, glyceryl stearate, glyceryl myristate, hydrogenated coco-glycerides and mixtures thereof.

15. Use of at least one benzotriazol derivative of formula (I) 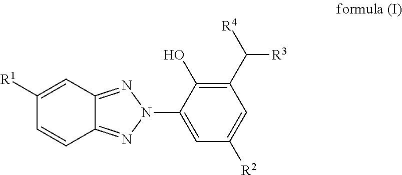
wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl; C₅₋₁₀cycloakl; C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl; preferably hydrogen for increasing the washability of organic UV-filter substances.

16. Method for increasing the washability of at least one organic UV-filter substance in a topical composition comprising a cosmetic solvent selected from the group of benzoate solvents, preferably C₁₂₋₁₅ alkyl benzoate, said method comprising the addition of at least one benzotriazol derivative of formula (I) 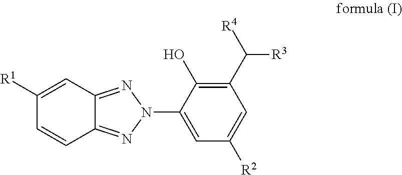
wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl, C₅₋₁₀cycloakl; C₁₋₂₀alkoxy or C₅₋₁₀cycloalkoxy; preferably C₅₋₁₅alkyl or C₅₋₁₅alkoxy; and
R⁴ is hydrogen or C₁₋₅alkyl; preferably hydrogen into said topical composition and observing or appreciating the result.

17. A method for the cosmetic treatment of keratinous substances, characterized in that a composition as defined in claim 1, is applied to the said keratinous substances.
